(19) United States
(12) Patent Application Publication
(10) Pub. No.: US 2009/0124817 A1
(43) Pub. Date: May 14, 2009

(54) Process for Preparing Amorphous Atorvastatin Calcium Nanoparticles

(75) Inventors: Sung-Joo Hwang, Seoul (KR); Min-Soo Kim, Gyeongsangnam-do (KR); Jeong-Soo Kim, Daejeon (KR); Ha-Seung Song, Daejeon (KR)

Correspondence Address: THE WEBB LAW FIRM, P.C., 700 KOPPERS BUILDING, 436 SEVENTH AVENUE, PITTSBURGH, PA, 15219, US

(73) Assignee: The Industry & Academic Cooperation in Chungnam National University, Daejeon (KR)

(21) Appl. No.: 11/937,920

(22) Filed: Nov. 9, 2007

(51) Int. Cl. C07D 207/337

(52) U.S. Cl. 548/537; 977/895

(57) ABSTRACT

The present invention relates to a method of preparing amorphous atorvastatin calcium nanoparticles using a supercritical fluid process. Specifically, the method comprising the steps of: a) dissolving atorvastatin calcium in an organic solvent with/without a hydrophilic additive to prepare a drug solution; b) introducing the drug solution and carbon dioxide into a reactor, which maintains carbon dioxide at supercritical conditions, to produce atorvastatin calcium particles; and c) introducing carbon dioxide into the reactor to wash the particles through removal of the remaining organic solvent. The amorphous atorvastatin calcium prepared according to the method of the present invention has a particle size of nanometer order, large surface area and high solubility shows improved bioavailability, and thus can be formulated as various preparations for oral administration.

### BACKGROUND OF THE INVENTION

1. Field of the Invention

The present invention relates to a novel process for preparing amorphous atorvastatin calcium nanoparticles.

2. Background of the Related Art

Atorvastatin represented by a formula of [R-(R*,R*)]-2-(4-fluorophenyl)-b,d-dihydroxy-5-(1-methyl-ethyl)-3-phenyl-4-[(phenylamino)carbonyl]-1H-pyrrole-1-heptanoic acid is a statin drug, which is an inhibitor of HMG-CoA reductase, the rate-limiting enzyme in cholesterol synthesis, and is useful as a therapeutic agent for hyperlipidemia and hypercholesterolemia.

Generally, atorvastatin is prepared in the form of calcium salts, because calcium salts can be easily formulated into oral administration forms such as tablets, capsules and powders. Atorvastatin is currently commercially available in the form of hemi-calcium trihydrate under the trade name “LIPITOR”.

Atorvastatin can exist in an amorphous form or in one of the crystalline forms (Form I, Form II, Form III and Form IV). U.S. Pat. No. 5,969,156 discloses crystalline Form I atorvastatin and hydrates thereof crystalline Form II atorvastatin and hydrates thereof and crystalline Form IV atorvastatin and hydrates thereof, and U.S. Pat. No. 6,121,461 discloses crystalline Form III atorvastatin hydrate. U.S. Pat. No. 6,087,511 discloses amorphous atorvastatin in the form of hydrates and anhydrides.

It is known that the amorphous forms in a number of pharmaceutical substances exhibit different dissolution characteristics and bioavailability patterns compared to the crystalline forms (Konno T., Chem Pharm Bull., 1990, 38: 2003-2007) and the particle size is one of important factors affecting the bioavailability. For some therapeutic indications, the bioavailability is one of the key parameters determining the form of the substance to be used in a pharmaceutical formulation. In the case of atorvastatin, amorphous forms have many advantages in terms of bioavailability compared to crystalline forms, and thus there is a need for a method for preparing amorphous atorvastatin having a particle size of nanometer order.

U.S. Pat. No. 6,087,511 and U.S. Pat. No. 6,274,740 disclose a method of preparing amorphous atorvastatin by converting crystalline atorvastatin calcium into amorphous atorvastatin. Specifically, amorphous atorvastatin calcium is prepared by dissolving crystalline Form I atorvastatin in a non-hydroxyl solvent such as tetrahydrofuran or tetrahydrofuran-toluene and removing the solvent by vacuum drying or spray drying. However, the amorphous atorvastatin calcium prepared according to this method has problems in that it is prepared in the form of brittle foams, and an excessively long drying time is required for the removal of the solvent, making it difficult to actually apply the method.

WO 01/28999 discloses a method of preparing amorphous atorvastatin calcium by recrystallization of crude atorvastatin, which comprises dissolving crude atorvastatin calcium in a lower alkanol under heating and isolating the amorphous atorvastatin calcium precipitated after cooling. However, there is a problem in that a large amount of alcohol is required.

### SUMMARY OF THE INVENTION

It is an object of the present invention to provide a method of preparing amorphous, nanometer-sized atorvastatin calcium fine particles with high purity in a simple and environment-friendly manner.

Thus, the present invention provides a method of preparing amorphous atorvastatin calcium nanoparticles using supercritical carbon dioxide. Specifically, the present invention provides a method for preparing amorphous atorvastatin calcium nanoparticles, the method comprising the steps of: dissolving atorvastatin calcium in an organic solvent to prepare a drug solution; b) introducing the drug solution and carbon dioxide into a reactor, which maintains carbon dioxide at supercritical conditions, to produce particles; c) introducing carbon dioxide into the reactor to wash the particles through removal of the remaining organic solvent.

### BRIEF DESCRIPTION OF THE DRAWINGS

The above and other objects, features and advantages of the present invention will be more clearly understood from the following detailed description taken in conjunction with the accompanying drawings, in which:

FIG. 1 shows a X-ray diffractogram of starting material atorvastatin calcium;

FIGS. 2 to 4 show X-ray diffractograms of atorvastatin calcium particles prepared using the supercritical fluid process of the present invention in Examples 2, 18 and 23, respectively;

FIG. 5 shows a X-ray diffractogram of atorvastatin calcium prepared using a spray drying method in Comparative Example 1;

FIG. 6 is a SEM photograph of starting material atorvastatin calcium;

FIGS. 7 to 9 are SEM photographs of atorvastatin calcium particles prepared using the supercritical fluid process of the present invention in Examples 18, 24 and 27, respectively;

FIG. 10 is a SEM photograph prepared using a spray drying method in Comparative Example 1;

FIG. 11 shows the TGA results of starting material atorvastatin calcium;

FIG. 12 shows the TGA results of atorvastatin calcium particles prepared in Example 2 using the supercritical fluid process of the present invention;

FIG. 13 shows the comparison of intrinsic dissolution rate between starting material atorvastatin calcium, and atorvastatin calcium particles prepared in Examples 2, 9 and 18 using the supercritical fluid process of the present invention;

FIG. 14 shows the comparison of plasma concentration after the administration of starting material atorvastatin calcium, a commercial product, atorvastatin calcium prepared in Comparative Example 1 using a spray drying method, and atorvastatin calcium particles prepared in Examples 18 and 25 using the supercritical fluid process of the present invention; and

FIG. 15 shows the comparison of kinetic solubility between stating material atorvastatin calcium, and atorvastatin calcium particles prepared in Examples 2, 41, 42, 44, 45 and 46 using the supercritical fluid process of the present invention.

### DETAILED DESCRIPTION OF THE PREFERRED EMBODIMENT

As used herein, the term “amorphous” refers to a solid form of a molecule that is not crystalline. An amorphous solid does not show a definitive X-ray diffraction pattern with sharp maxima.

As used herein, the term “supercritical fluid” refers to an incompressible fluid at or above its critical temperature and pressure and has features that are uniquely different from those of conventional organic solvent. Namely, a supercritical fluid has the advantage properties of both liquid and gas, e.g., a high density close to that of a liquid, a low viscosity and high diffusion coefficient close to those of gas, and a very low surface tension.

Since the density of a supercritical fluid can be continuously changed from a sparse state like an ideal gas to a highly dense state like a liquid, its physicochemical properties at equilibrium (e.g., solubility), mass transfer characteristics (e.g., viscosity, diffusion coefficient and thermal conductivity) and molecular clustering state of the fluid can be regulated. Therefore, by regulating the properties of a supercritical fluid, it is possible to obtain a solvent having properties which correspond to a combination of those of several solvents. In the present invention, supercritical carbon dioxide is used as supercritical fluid. Carbon dioxide has a low critical temperature of 31.1□ so that it can be used for a thermally unstable substance such as a protein drug. Furthermore, since carbon dioxide is nontoxic incombustible, inexpensive and recyclable, it is environmentally friendly and can be advantageously used in a process of preparing medical products. As used herein, the term “supercritical carbon dioxide” refers to carbon dioxide at or above its critical temperature and pressure.

The present invention provides a method of preparing amorphous atorvastatin calcium nanoparticles using supercritical carbon dioxide. Specifically, the present invention provides a method for preparing amorphous atorvastatin calcium nanoparticles, the method comprising the steps of; a) dissolving atorvastatin calcium in an organic solvent with/without a hydrophilic additive to prepare a drug solution; b) introducing the drug solution and carbon dioxide into a reactor, which maintains carbon dioxide at supercritical conditions, to produce particles; c) introducing carbon dioxide into the reactor to wash the particles through removal of the remaining organic solvent.

Hereinafter, each step of the preparation method according to the present invention will be described in further detail.

Step a): Preparation of Crystalline Atorvastatin Calcium Drug Solution

This step is a step of dissolving atorvastatin calcium in an organic solvent to prepare a drug solution.

Atorvastatin used as the starting material may be in a crystalline form and a mixed crystalline/amorphous form. A crystalline form is preferred.

As the organic solvent, a solvent capable of freely dissolving atorvastatin is used. The term “freely dissolving” means that atorvastatin can be completely dissolved in any solvent, that is, does not leave any solid. As the organic solvent, a C1-C6 lower alcohol such as methanol or ethanol, acetone, dimethyl sulfoxide, n-methylpyrrolidone or tetrahydrofuran can be used, and a C1-C6 lower alcohol is preferred. Acetone or tetrahydrofuran is particularly preferred. The drug solution is preferably prepared at a concentration of 10-500 mg/ml.

Also, to further increase yield and solubility and to inhibit the conversion of amorphous form to crystalline form, a hydrophilic additive can be added alone or in a mixture to prepare the drug solution. Particular examples of the hydrophilic additive that may be used in the present invention include: cellulose derivatives such as hydroxypropylmethyl cellulose (HPMC), hydroxypropyl cellulose (HPC), hydroxyethyl cellulose (HEC) or sodium carboxymethyl cellulose (Na—CMC); polyvinyl pyrrolidones such as polyvinyl pyrrolidone K-30 (PVP K-30); copolymers of vinyl pyrrolidone and vinyl acetate, such as polyvinyl pyrrolidone vinyl acetate (PV VA-64); polyethylene glycol (PEG); polyethylene glycol-derivatized vitamin E such as vitamin E TPGS; poloxamers; polyglycolated glycerides such as Gelucire 44/14; polyvinyl alcohols (PVA); cyclodextrin (e.g. β-CD); polymethylmethacrylate derivatives; chitin; chitosan and derivatives thereof; alginic acid and alkali salts and metal salts thereof; and polysaccharides such as caraginaan gum, tragacanth gum, agar, arabic gum, guar gum or xanthan gum. Particularly, hydroxypropylmethyl cellulose (HPMC), polyvinyl pyrrolidone, copolymers of vinyl pyrrolidone and vinyl acetate, polyethlylene glycol (PEG), polyethlylene gylcol (PEG)—derivatized vitamin E, poloxamers, polyglycolated glycerides are preferred. The hydrophilic additive can be used in an amount of 0.1-90 wt %, and preferably 1-50 wt %, based on the total weight of atorvastatin calcium.

Step b): Production of Particles Using Supercritical Fluid Process

This step is a step comprising: introducing carbon dioxide into a reactor, heating and pressurizing carbon dioxide to a temperature and pressure above its critical points (31.06□ and 73.8 bar) to maintain carbon dioxide at supercritical conditions, and then introducing the drug solution and carbon dioxide into the reactor to produce particles.

The reactor is preferably made of stainless steel such that it can resist pressure. During carbon dioxide pressurization, a syringe pump is used to maintain constant pressure and know a precise injection amount of carbon dioxide, and a constant-temperature water bath or an automatic temperature controller is used to maintain constant temperature. Herein, the inside of the reactor is preferably maintained at a temperature of 35-80□ and a pressure of 80-200 bar.

The drug solution is introduced by injecting it at constant rate using a small-sized speed-controllable liquid pump. In order to prevent the supercritical carbon dioxide in the reactor from being saturated and to efficiently inject the drug solution into the supercritical carbon dioxide through a nozzle, the drug solution and carbon dioxide are introduced at the same time through the same nozzle at constant rate. In order to prevent the nozzle from being plugged, about 5 ml of a solvent used in the preparation of the drug solution is preferably introduced before the injection of the drug solution. The ratio of introduction rate of the drug solution to introduction rate of carbon dioxide is preferably maintained at 1:10-1:120 based on weight.

The organic solvent in the injected drug solution is rapidly mixed with supercritical fluid, and atorvastatin calcium is over-saturated and precipitated to produce amorphous atorvastatin calcium particles.

Step c): Removal of Remaining Organic Solvent Using Supercritical Fluid

This step is a process of washing the prepared amorphous atorvastatin calcium particles and is performed by introducing additional carbon dioxide to remove the remaining organic solvent. To maintain the inside of the reactor at constant pressure, a mixed gas of carbon dioxide and solvent in the reactor is discharged through the outlet at the same rate as the introduction rate of carbon dioxide. Herein, to maintain the reactor at constant pressure, a back pressure regulator is connected to the reactor. A 0.45-μm membrane filter is used in the outlet to prevent the particles from coming out.

If the washing process is not sufficient, the remaining organic solvent will be re-extracted upon depressurization to wet the precipitated particles so as to form aggregates and show solvent toxicity. For this reason, the washing process is sufficiently carried out until the remaining organic solvent can be completely removed.

The amount of carbon dioxide used in the washing process is preferably 30-80 times of the volume of the reactor.

After washing, the reactor is depressurized to discharge supercritical carbon dioxide slowly. Then, the prepared amorphous atorvastatin calcium particles are recovered from the wall or bottom of the reactor.

The atorvastatin calcium prepared according to the method of the present invention is amorphous, has an average particle size of nanometer order, shows high dissolution rate due to its increased specific surface area and amorphous form, and shows increased bioavailability. Also, supercritical carbon dioxide used in the present invention is nontoxic, incombustible, inexpensive and recyclable, and thus the method of the present invention is useful for the mass production of amorphous atorvastatin calcium nanoparticles in an economic and environmental-friendly manner.

Hereinafter, the present invention will be described in further detail with reference to examples, but these examples are not to be construed to limit the scope of the present invention.

### Examples 1-40

### Preparation 1 of Amorphous Atorvastatin Calcium Particles by Supercritical Fluid Process

TABLE 1

| | | | Supercritical process conditions | | | |
|---|---|---|---|---|---|---|
| | | | | | | Drug |
| | | | | | CO₂ | solution |
| | Solution conditions | | Temperature | Pressure | Injection | injection |
| | Atorvastatin | Solvent | (° C.) | (bar) | rate | rate |
| Example 1 | 50 mg/ml | Methanol | 40 | 100 | 45 g/min | 0.5 g/min |
| Example 2 | 50 mg/ml | Methanol | 40 | 120 | 45 g/min | 0.5 g/min |
| Example 3 | 50 mg/ml | Methanol | 40 | 150 | 45 g/min | 0.5 g/min |
| Example 4 | 50 mg/ml | Methanol | 40 | 180 | 45 g/min | 0.5 g/min |
| Example 5 | 50 mg/ml | Methanol | 50 | 120 | 45 g/min | 0.5 g/min |
| Example 6 | 50 mg/ml | Methanol | 60 | 120 | 45 g/min | 0.5 g/min |
| Example 7 | 10 mg/ml | Methanol | 40 | 120 | 45 g/min | 0.5 g/min |
| Example 8 | 25 mg/ml | Methanol | 40 | 120 | 45 g/min | 0.5 g/min |
| Example 9 | 100 mg/ml | Methanol | 40 | 120 | 45 g/min | 0.5 g/min |
| Example 10 | 150 mg/ml | Methanol | 40 | 120 | 45 g/min | 0.5 g/min |
| Example 11 | 50 mg/ml | Methanol | 40 | 120 | 30 g/min | 0.5 g/min |
| Example 12 | 50 mg/ml | Methanol | 40 | 120 | 30 g/min | 1 g/min |
| Example 13 | 50 mg/ml | Methanol | 40 | 120 | 60 g/min | 1 g/min |
| Example 14 | 50 mg/ml | Methanol | 40 | 120 | 60 g/min | 0.5 g/min |
| Example 15 | 50 mg/ml | Methanol | 40 | 120 | 45 g/min | 1 g/min |
| Example 16 | 10 mg/ml | Tetrahydrofuran | 40 | 120 | 45 g/min | 0.5 g/min |
| Example 17 | 50 mg/ml | Tetrahydrofuran | 40 | 120 | 45 g/min | 0.5 g/min |
| Example 18 | 100 mg/ml | Tetrahydrofuran | 40 | 120 | 45 g/min | 0.5 g/min |
| Example 19 | 150 mg/ml | Tetrahydrofuran | 40 | 120 | 45 g/min | 0.5 g/min |
| Example 20 | 200 mg/ml | Tetrahydrofuran | 40 | 120 | 45 g/min | 0.5 g/min |
| Example 21 | 300 mg/ml | Tetrahydrofuran | 40 | 120 | 45 g/min | 0.5 g/min |
| Example 22 | 100 mg/ml | Tetrahydrofuran | 40 | 200 | 45 g/min | 0.5 g/min |
| Example 23 | 100 mg/ml | Tetrahydrofuran | 80 | 120 | 45 g/min | 0.5 g/min |
| Example 24 | 50 mg/ml | Acetone | 40 | 120 | 45 g/min | 0.5 g/min |
| Example 25 | 100 mg/ml | Acetone | 40 | 120 | 45 g/min | 0.5 g/min |
| Example 26 | 200 mg/ml | Acetone | 40 | 120 | 45 g/min | 0.5 g/min |
| Example 27 | 300 mg/ml | Acetone | 40 | 120 | 45 g/min | 0.5 g/min |
| Example 28 | 100 mg/ml | Acetone | 35 | 120 | 45 g/min | 0.5 g/min |
| Example 29 | 100 mg/ml | Acetone | 40 | 180 | 45 g/min | 0.5 g/min |
| Example 30 | 50 mg/ml | Acetone | 40 | 120 | 45 g/min | 0.5 g/min |
| Example 31 | 400 mg/ml | Acetone | 40 | 120 | 45 g/min | 0.5 g/min |
| Example 32 | 50 mg/ml | DMSO | 40 | 120 | 45 g/min | 0.5 g/min |
| Example 33 | 100 mg/ml | DMSO | 40 | 120 | 45 g/min | 0.5 g/min |
| Example 34 | 200 mg/ml | DMSO | 40 | 120 | 45 g/min | 0.5 g/min |
| Example 35 | 50 mg/ml | NMP | 40 | 120 | 45 g/min | 0.5 g/min |
| Example 36 | 100 mg/ml | NMP | 40 | 120 | 45 g/min | 0.5 g/min |
| Example 37 | 200 mg/ml | NMP | 40 | 120 | 45 g/min | 0.5 g/min |
| Example 38 | 10 mg/ml | Ethanol | 40 | 120 | 45 g/min | 0.5 g/min |
| Example 39 | 30 mg/ml | Ethanol | 40 | 120 | 45 g/min | 0.5 g/min |
| Example 40 | 20 mg/ml | Acetonitrile | 40 | 120 | 45 g/min | 0.5 g/min |

DMSO: dimethyl sulfoxide; and
NMP: N-methyl pyrrolidone

Starting material atorvastatin calcium was dissolved in each of solvents set forth in Table 1 above to prepare a drug solution. Carbon dioxide was introduced into a reactor having an inner diameter of 9 cm, a height of 30 cm and a volume of 1908 cm3 and adjusted to the temperature and pressure set forth in Table 1 to maintain supercritical carbon dioxide at equilibrium. About 5 ml of a solvent used in the preparation of the drug solution was injected into the reactor by means of a liquid pump, and then the prepared drug solution together with carbon dioxide was introduced into the reactor at the constant rate set forth in Table 1. While the drug solution was injected through a nozzle in the reactor, particles were produced. While carbon dioxide was injected into the reactor, gas such as carbon dioxide in the reactor was discharged at the same rate through the outlet using a back pressure regulator to remove the organic solvent dissolved in the supercritical carbon dioxide. The washing process was carried out using about 15,000 ml of carbon dioxide. After completion of the washing process, carbon dioxide in the reactor was completely discharged and the produced atorvastatin calcium particles were collected from the wall and bottom of the reactor.

### Examples 41-48

### Preparation 2 of Amorphous Atorvastatin Calcium Particles by Supercritical Fluid Process

TABLE 2

| | Example | Example | Example | Example | Example | Example | Example | Example | Example | Example |
|---|---|---|---|---|---|---|---|---|---|---|
| | 41 | 42 | 43 | 44 | 45 | 46 | 48 | 49 | 50 | 51 |
| Atorvastat | 4.5 g | 4 g | 3 g | 4 g | 4 g | 4 g | 4 g | 4 g | 4 g | 4 g |
| in calcium | | | | | | | | | | |
| HPMC | 0.5 g | 1 g | 2 g | — | — | — | — | — | — | — |
| HPC | — | — | — | 1 g | — | — | — | — | — | — |
| PVP K30 | — | — | — | — | 1 g | — | — | — | — | — |
| PVP | — | — | — | — | — | 1 g | — | — | — | — |
| VA64 | | | | | | | | | | |
| PEG6000 | — | — | — | — | — | — | 1 g | — | — | — |
| Gelucire | — | — | — | — | — | — | — | 1 g | — | — |
| 44/14 | | | | | | | | | | |
| Poloxamer | — | — | — | — | — | — | — | — | 1 g | — |
| 407 | | | | | | | | | | |
| Vitamin E | — | — | — | — | — | — | — | — | — | 1 g |
| TPGS | | | | | | | | | | |
| Tetrahydrofuran | 50 ml | 50 ml | 50 ml | 50 ml | — | — | — | — | — | — |
| Methanol | — | — | — | — | 50 ml | 50 ml | 50 ml | 50 ml | 50 ml | 50 ml |

A mixed solution containing atorvastatin calcium and a hydrophilic additive in the ratio and amount as shown in following Table 2 was provided. Then, amorphous atorvastatin calcium nanoparticles containing a hydrophilic additive were prepared under the same conditions of the supercritical fluid process as described in Example 2.

### Comparative Example 1

### Preparation of Amorphous Atorvastatin Calcium by Spray-Drying

Atorvastatin calcium was dissolved in tetrahydrofuran to obtain clear solutions. Spray drying was carried out using a laboratory scale spray dryer (SD 1000, Eyela, Japan) under following set of conditions: drug solution concentration: 100 mg/ml; inlet temperature: 75° C.; outlet temperature: 62-65° C.; feed rate: 3 ml/min; atomization air pressure: 10 kPa; and drying air flow rate: 0.70 m3/min.

### Test Example 1

### Powder XRD Analysis

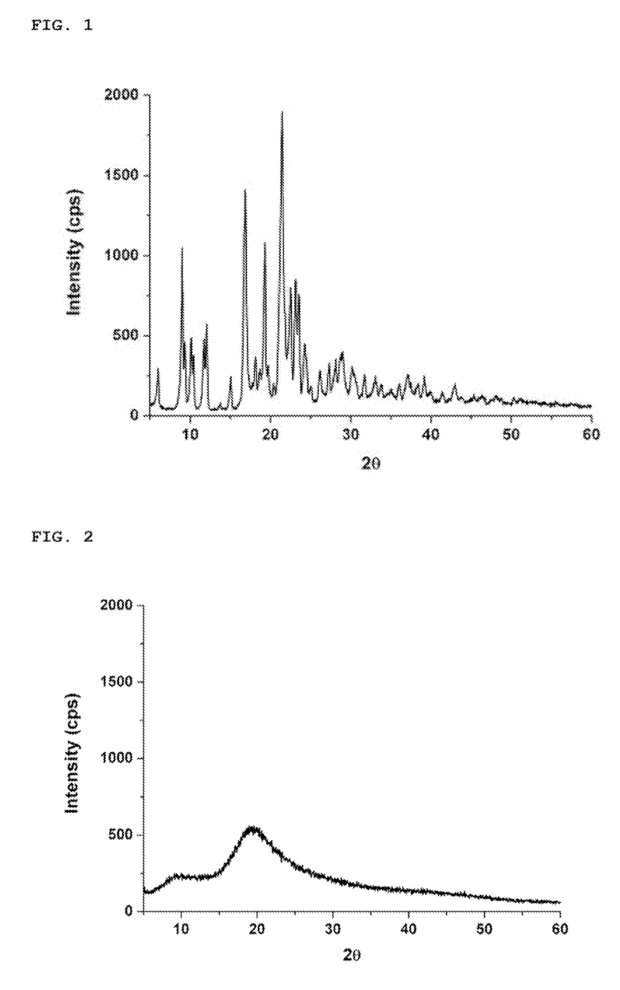

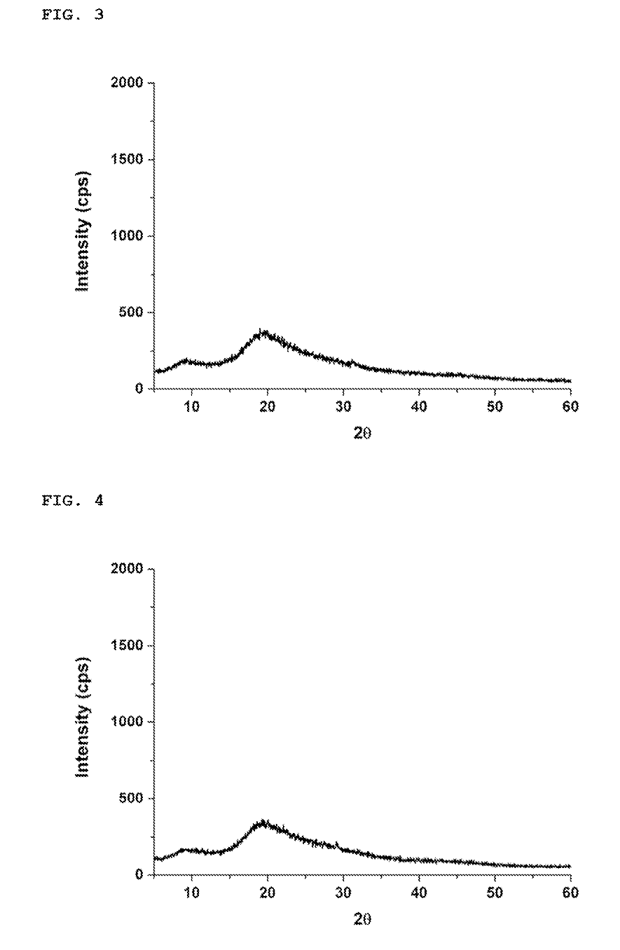

Starting material atorvastatin calcium powder, the atorvastatin calcium particles prepared in Examples 2, 18 and 23 using the supercritical fluid process of the present invention, and the atorvastatin calcium prepared in Comparative Example 1 using the spray-drying method, were observed for crystallinity using an X-ray diffraction analyzer, and the observation results are shown in FIGS. 1 to 5, respectively.

A trace amount of each of the samples was placed on the sample holder of a powder X-ray diffraction analyzer (Rigaku (Japan), D/MAX-2200). The analysis was performed using Ni-filtered Cu—Kα radiation at a step size of 0.02°, a step rate of 1.2/sec and an angle (2θ) of 5°-60°.

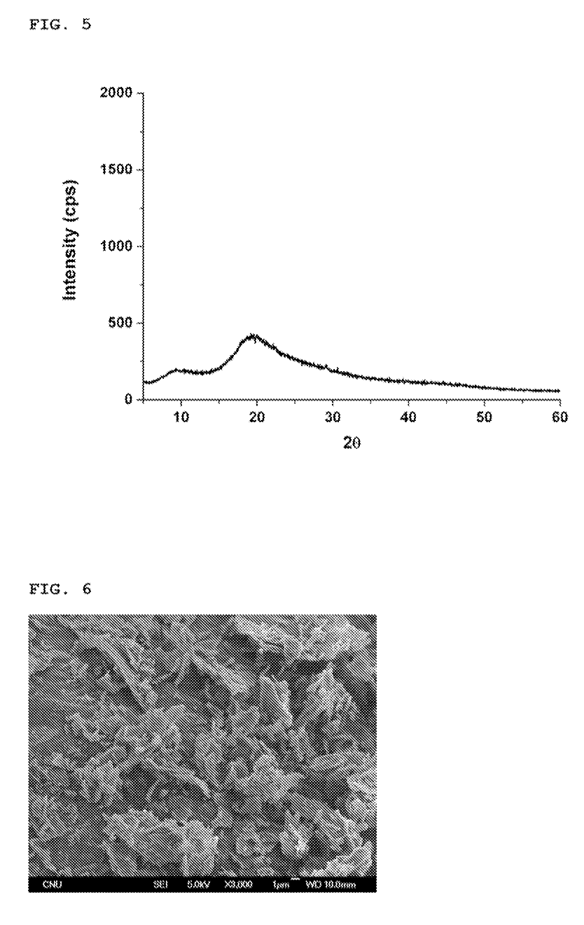

From FIG. 1, it can be seen that the starting material atorvastatin calcium was crystalline. As can be seen in FIGS. 2 to 4, the atorvastatin calcium particles prepared in Examples 2, 18 and 23 were amorphous, because they showed round curves and had no sharp peaks. As can be seen in FIG. 5, the particles prepared in Comparative Example 1 were also amorphous.

### Test Example 2

### Scanning Electron Microscope (SEM) Observation of Amorphous Atorvastatin Calcium Particles Prepared Using Supercritical Fluid Process

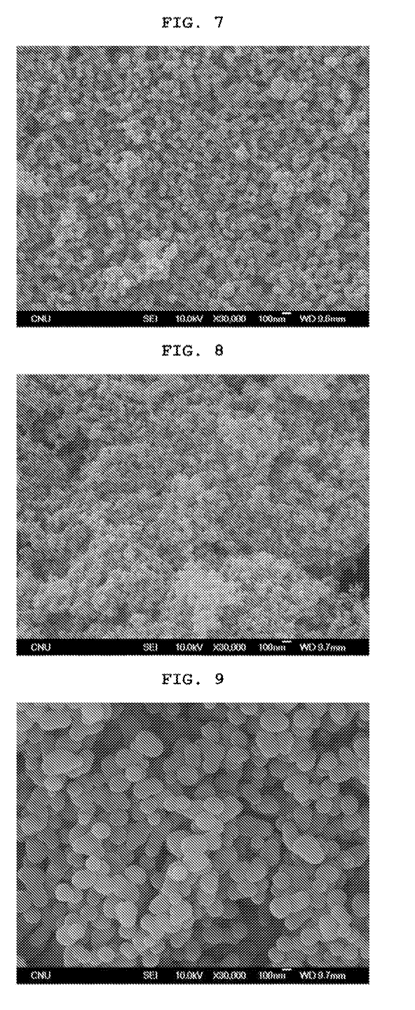

Starting material atorvastatin calcium powder, the atorvastatin calcium particles prepared in Examples 18, 24 and 27 using the supercritical fluid process of the present invention, and the atorvastatin calcium in Comparative Example 1 using the spray-drying method, were observed using SEM (Scanning Electron Microscopy). The observation results are shown in FIGS. 6 to 10. The observation was performed using JSM-7000 (Jeol Ltd., Japan) at an accelerating voltage of 10 kV.

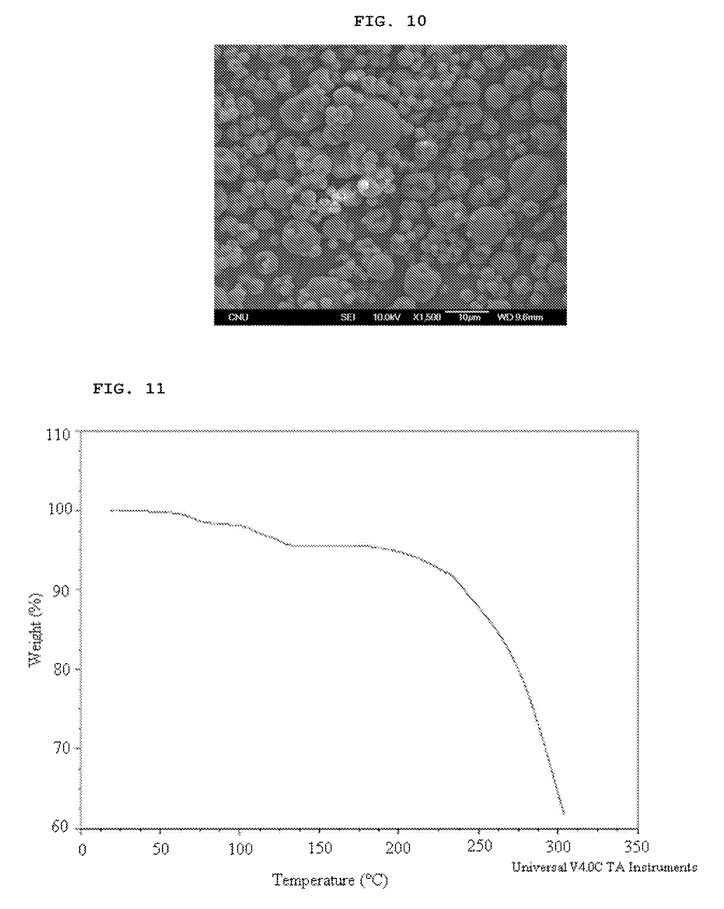

As shown in FIG. 6, the starting material atorvastatin particles are non-uniform particles having a particle size larger than the micrometer scale. Also, as can be seen in FIG. 10, the atorvastatin calcium prepared in Comparative Example 1 using the spray-drying method were non-uniform and showed a particle size larger than the micrometer scale. On the other hand, as can be seen in FIGS. 7 to 9, the amorphous atorvastatin calcium particles prepared in Examples 18, 24 and 27 using the supercritical fluid process of the present invention were nanometer-sized, uniform particles.

### Test Example 3

### TGA Analysis of Amorphous Atorvastatin Calcium Prepared Using Supercritical Fluid Process

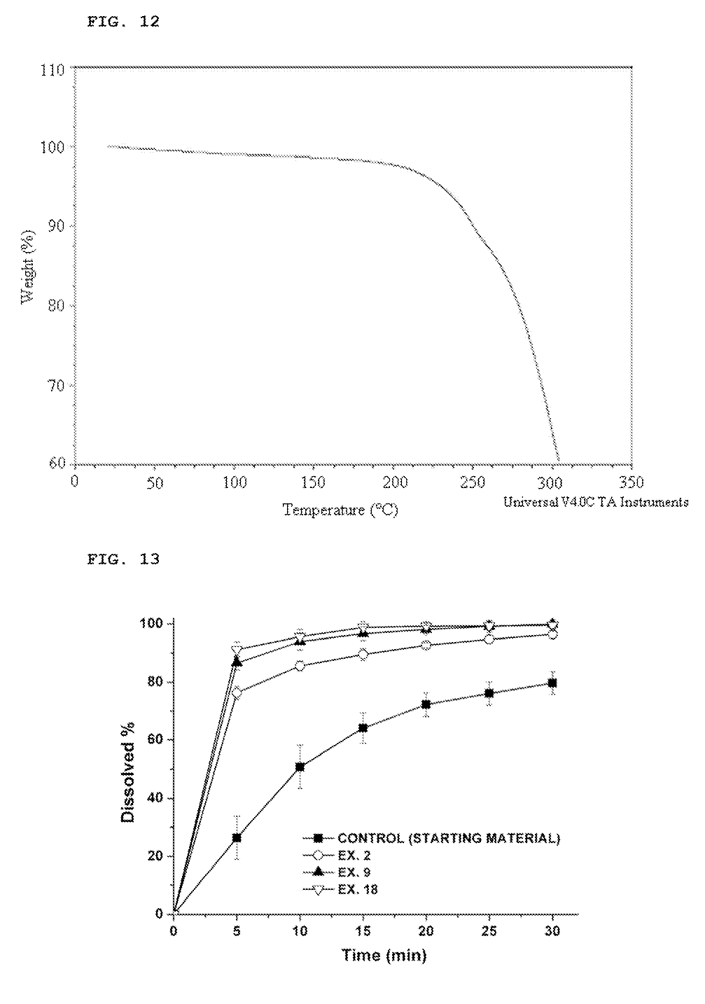

Starting material atorvastatin calcium powder and the atorvastatin calcium particles prepared in Example 2 using the supercritical fluid process of the present invention were analyzed by TGA, and the analysis results are shown in FIGS. 11 and 12, respectively.

Thermal gravimetric analysis (TGA) was performed using a TA instruments (USA) TGA 2950 Thermogravimetrical Analyzer, The experiment was performed with a heating rate of 5° C./min using nitrogen flow (50 ml/min) and the samples weighed (approximately 5 mg) in open aluminum pans and the percentage weight loss of the samples was monitored from 20 to 300° C.

As a result as can be seen in FIG. 11, the starting material atorvastatin calcium showed a change of about 4.5% in weight due to the loss of water of three molecules. However, as can be seen in FIG. 12, the atorvastatin calcium particles prepared in Example 2 using the supercritical fluid process did not show a change in weight. That is, anhydrous amorphous atorvastatin calcium particles were prepared using the supercritical fluid process.

### Test Example 4

### Analysis of Particle Size and Specific Surface Area of Amorphous Atorvastatin Calcium Prepared by Supercritical Fluid Process

Starting material atorvastatin calcium powder, the amorphous atorvastatin calcium particles prepared in Examples 2, 9, 10, 18 and 25, and the amorphous atorvastatin calcium prepared in Comparative Example 1, were analyzed by DLS, and the analysis results are shown in Table 3.

The particle size and particle size distribution of samples were determined by dynamic light scattering (DLS) using electrophoretic light scattering spectrophotometer (ELS-8000, Otsuka Electronics, Japan) at a fixed angle of 90° and at room temperature. The samples were dispersed in mineral oil (Macrol 52, Exxon Mobil Co., USA) and sonicated before measurement. The particle size and particle size distribution of samples were also determined with a Sympatec laser diffraction analyzer (HELOS/RODOS, Clausthal-Zellerfeld, Germany) consisted of a laser sensor HELOS and a RODOS dry-powder air-dispersion system. The specific surface area was determined using the gas adsorption method. Calculation is based on the BET equation. Surface Area Analyzer ASAP 2010 (Micromeritics Instrument Corporation, USA) was used.

TABLE 3

| | Mean particle size | | Specific surface area (m²/g) |
|---|---|---|---|
| Example 2 | 179 | nm | 90.28 |
| Example 9 | 265 | nm | 47.82 |
| Example 10 | 727 | nm | 30.88 |
| Example 18 | 155 | nm | 79.78 |
| Example 25 | 65 | nm | 120.35 |
| Comparative Example 1 | 7.31 | μm | 0.95 |
| Starting material | 3.87 | μm | 14.56 |

As shown in Table 3, the amorphous atorvastatin calcium particles prepared according to the method of the present invention were nanoparticles having a particle size of below 1 μm, preferably below 100 nm and showed a high specific surface area due to this reduction in particle size.

### Test Example 5

### Measurement of Intrinsic Dissolution Rate of Atorvastatin Calcium Particles Prepared by Supercritical Fluid Process

Starting material atorvastatin calcium powder and the amorphous atorvastatin calcium particles prepared in Examples 2 and 10 were measured for intrinsic dissolution rate (IDR), and the measurement results are shown in Table 4.

Intrinsic dissolution rate (IDR) studies were performed by the stationary disc (0.5 cm2 surface area, Distek Inc., USA) method using the USP XXIV paddle method using VK 7000 dissolution testing station and VK 750d heater/circulator (Vankel, USA). Discs were prepared compressing 80 mg of powder (as atorvastatin) in a Perkin Elmer hydraulic press, for 1 mm under 5 t compression. Analysis of the compressed discs by DSC confirmed that the crystal form of the original powder was retained following the compression procedure. All dissolution runs were carried out in triplicate, under sink conditions (distilled water containing 1% SLS). Then, 4 ml of aliquot samples were withdrawn in certain time intervals and filtered using a 0.22-μm nylon syringe filter. At each sampling time, an equal volume of the test medium was replaced. Filtered samples were appropriately diluted with methanol and assayed for drug concentration by HPLC. Chromatographic analyses were performed on a Waters HPLC system consisting of a pump (Model 600), an auto-sampler (Model 717 plus), UV detector (Model 486 Tunable Absorbance Detector),

Column: Xterra, 5 μm, 4.6 mm 250 mm

UV wavelength: 245 nm

Flow rate: 1.0 ml/min

Mobile phase: 60:40 mixture of acetonitrile: 50 mM sodium acetate in water, where the pH was adjusted to 4.0 with glacial acetic acid

Injection volume: 20 ul

The linear portion of each dissolution profile, i.e. before depletion of the disc and alteration of its surface area, was used to derive the intrinsic dissolution rate.

The test results are shown in Table 4 below.

TABLE 4

| | Intrinsic dissolution rate (mg/cm²/min) |
|---|---|
| Example 2 | 0.279 |
| Example 10 | 0.278 |
| Starting material | 0.086 |

As shown in Table 4, the amorphous atorvastatin showed an intrinsic dissolution rate about 3.2-fold higher than that of the crystalline atorvastatin.

### Test Example 6

### Measurement of Dissolution Rate of Amorphous Atorvastatin Calcium Particles Prepared by Supercritical Fluid Process

Starting material atorvastatin calcium powder and the amorphous atorvastatin calcium particles prepared in Examples 2, 9 and 18 were measured for dissolution rate, and the measurement results are shown in FIG. 13.

Dissolution studies were performed according to the USP XXIV paddle method using VK 7000 dissolution testing station and VK 750d heater/circulator (Vankel, USA). The stirring speed used was 50 rpm, and the temperature was maintained at 37±0.1° C. Each test was carried out in 900 ml of distilled water. Accurately weighted samples containing the equivalent of 10 mg atorvastatin were placed in the dissolution medium. Then, 4 ml of aliquot samples were withdrawn in certain time intervals and filtered using a 0.22-μm nylon syringe filter. At each sampling time, an equal volume of the test medium was replaced. Filtered samples were appropriately diluted with methanol and assayed for drug concentration by HPLC.

As shown in FIG. 13, the atorvastatin calcium particles prepared using the supercritical fluid process showed increased dissolution rate compared to the starting material atorvastatin. Specifically, at 5 minutes after the start of dissolution, the starting material atorvastatin showed a dissolution rate of about 25%, whereas the amorphous atorvastatin calcium particles of Examples showed a high dissolution rate of more than about 85%. This increase in dissolution rate is attributable to increased intrinsic dissolution rate and increased specific surface area caused by a small particle size of nanometer order.

### Test Example 7

### Animal Test of Amorphous Atorvastatin Calcium Particles Prepared by Supercritical Fluid Process

To evaluate the bioavailability of the amorphous atorvastatin calcium nanoparticles according to the present invention, the amorphous atorvastatin calcium nanoparticles according to Example 18 and Example 25 were used as a sample, while the amorphous atorvastatin calcium according to Comparative Example 1, the raw material (crystalline atorvastatin calcium) and commercial product (Lipitor® tablet) were used as control. As test animals, male Sprague-Dawley rats (body weight 220 g) were used. The rats were raised in a cage under constant conditions by using general solid feed for rats and by supplying water. The test animals were fasted for at least 24 hours for use in the following absorption test. During the fast period, the test animals were allowed to drink water freely. The sample or the control was administered to the rats in an effective amount of 25 mg per kg of body weight as expressed in terms of atorvastatin via an oral administration kit. Blood-gathering was performed before the administration and 15, 30, 45, 60, 90, 120, 240, 360 and 480 min after the administration from the femoral veins of the rats for test. To 200 μl of the blood plasma gathered as described above, 40 μl of an internal standard solution (100 ng/ml of methaqualone) and 800 μl of acetonitrile were added, followed by mixing. Then, the resultant turbid layer was collected, followed by centrifugation, evaporation and concentration under nitrogen flow. To the residue obtained thereby, 200 μl of methanol was added so that the residue was dissolved therein, and then LC/MS was performed under the following conditions to determine the concentration of atorvastatin.

Column: Kromasil C18 4.6 150 mm 5 um

UV wavelength: 270 nm

Flow rate: 1.5 ml/min

Mobile phase: 0.1 M ammonium acetate buffer (pH 4.0)/acetonitrile (1/1)

Injection volume: 5 ul

MS condition

ESI probe

Detection m/z: 559 (for target drug), 251 (for internal standard)

Detection gain: 1.50 kV

Nebulization Nitrogen flow: 1.5 L/min

CDL temperature: 250oC

Block temperature: 200oC

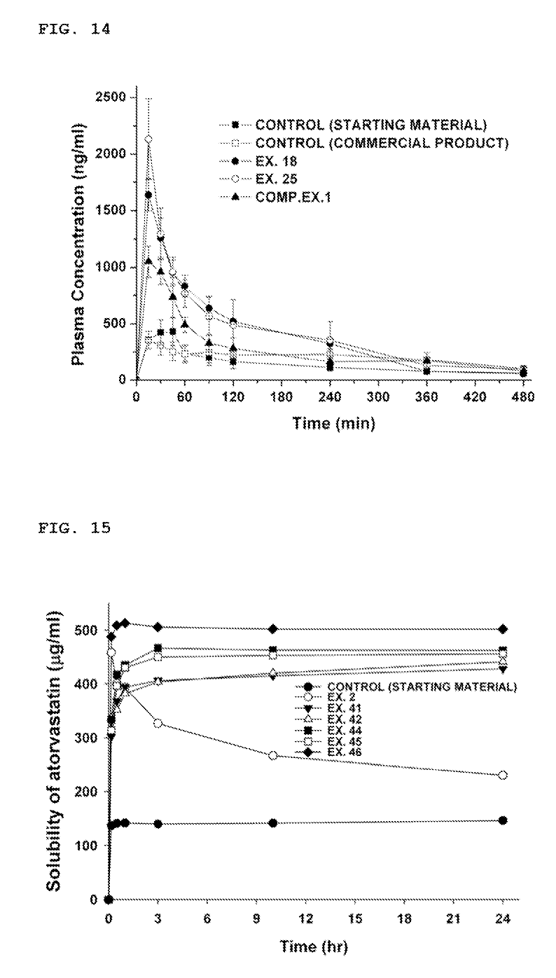

The test results are shown in FIG. 14 and Table 5.

TABLE 5

| | | | | Starting | Commercial |
|---|---|---|---|---|---|
| | EX. 18 | EX. 25 | COMP EX. 1 | material | product |
| AUC_{480 min} | 180964 ± 12020 | 194969 ± 24385 | 125994 ± 20352 | 67283 ± 12722 | 97279 ± 20523 |
| (ng/ml · min) | | | | | |
| Cₘₐₓ | 1637 ± 138 | 2133 ± 353 | 1050 ± 137 | 504 ± 66 | 367 ± 41 |
| Tₘₐₓ (min) | 15 | 15 | 15 | 34 ± 8 | 19 ± 8 |

AUC: area under the curve showing concentrations in blood by the time of 480 min after the final blood-gathering, as calculated by the trapezoidal rule.
Cₘₐₓ: maximum concentration in blood (the concentration at the time where the actual maximum concentration in blood appears).
Tₘₐₓ: time where the actual maximum concentration in blood appears.

As shown in FIG. 13 and Table 5, the AUC of the amorphous atorvastatin nanoparticles prepared in Examples 18 and 25 of the present invention was about 3-fold higher than that of the starting material and about 2-fold higher than that of the commercial product, and about 1.5-fold higher than that of the particles in Comparative Example 1. Such results suggest that not only the increase in intrinsic dissolution rate caused by an amorphous form, but also particle size, can have a great effect on bioavailability. That is, the atorvastatin particles prepared according to the present invention show, in addition to the advantages of existing amorphous atorvastatin, increased dissolution rate and improved bioavailability, due to a very fine particle size of less than 200 nm and increased specific surface area.

### Test Example 8

### Kinetic Solubility Test of Amorphous Atorvastatin Calcium Prepared by Supercritical Fluid Process

Starting material atorvastatin calcium powder and the amorphous atorvastatin calcium particles prepared in Examples 41, 42, 44, 45 and 46 were measured for kinetic solubility studies, and the measurement results are shown in FIG. 15. Excess solid (approximately 100 mg as atorvastatin) was placed with 200 ml water in a water-jacked vessel linked to a temperature controlled water bath held at 37±0.5° C. Solutions were agitated constantly by overhead stirrers at 200 rpm. Suitable aliquots were withdrawn in certain time intervals and filtered using a 0.11-μm nylon syringe filter. Filtered samples were diluted with methanol, and the concentration of atorvastatin was determined by HPLC.

As shown in FIG. 15, amorphous atorvastatin nanoparticles of Example 2 showed a supersaturation solubility about 3.2-fold higher than that of the starting material. However, as time passes by, its solubility was decreased. Such decrease of solubility is due crystallization to crystalline, as shown in FIG. 15, amorphous atorvastatin nanoparticles prepared in the present invention can maintain high supersaturation solubility by addition of hydrophilic additive.

### INDUSTRIAL APPLICABILITY

As described above, according to the method of the present invention, nanometer-sized, uniform amorphous atorvastatin calcium particles can be prepared in large amounts in an economic and environment-friendly manner. The atorvastatin calcium particles prepared according to the method of the present invention are amorphous, have a particle size of nanometer order, and thus show improved dissolution properties, leading to high bioavailability.

Although the preferred embodiment of the present invention has been described for illustrative purposes, those skilled in the art will appreciate that various modifications, additions and substitutions are possible, without departing from the scope and spirit of the invention as disclosed in the accompanying claims.

The invention claimed is:

1. A method for preparing amorphous atorvastatin calcium nanoparticles having a average particle size of below 1 μm, comprising the steps of
a) dissolving atorvastatin calcium in an organic solvent with/without a hydrophilic additive to prepare a drug solution;
b) introducing the drug solution and carbon dioxide into a reactor, which maintains carbon dioxide at supercritical conditions, to produce particles; and
c) introducing carbon dioxide into the reactor to wash the particles through removal of the remaining organic solvent.

2. Amorphous atorvastatin calcium nanoparticles according to claim 1, wherein the hydrophilic additive is selected from the group consisting of hydropropylmethylcellulose (HPMC), polyvinyl pyrrolidone, copolymers of vinyl pyrrolidone and vinyl acetate, polyethylene glycol (PEG), polyethylene glycol-derivatized vitamin E, poloxamers and polyglycolated glycerides.

3. Amorphous atorvastatin calcium nanoparticles according to claim 1, wherein the hydrophilic additive is used in an amount of 0.1˜90 wt % based on the total weight of atorvastatin calcium.

4. The method of claim 1, wherein the atorvastatin calcium used in the step a) is crystalline atorvastatin.

5. The method of claim 1, wherein the organic solvent in the step a) is at least one selected from the group consisting of C1-C6 alcohols, acetone, dimethyl sulfoxide, n-methylpyrrolidone and tetrahydrofuran.

6. The method of claim 1, wherein the drug solution in the step a) is prepared at a concentration of 10-500 mg/ml.

7. The method of claim 1, wherein conditions inside of the reactor are maintained at a temperature of 35-80° C. and a pressure of 80-200 bar.

8. The method of claim 1, wherein the ratio of introduction rate of the drug solution to introduction rate of carbon dioxide in the step b) is 1:10-120.
